(19) United States
(12) Patent Application Publication
(10) Pub. No.: US 2006/0127426 A1
(43) Pub. Date: Jun. 15, 2006

(54) Cosmetic effervescent cleansing pillow with rupturable packet

(75) Inventors: Michael Edward Ross, Naperville, IL (US); Dennis Irwin Steiger, New Milford, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division of Conopco, Inc.

(21) Appl. No.: 11/010,253

(22) Filed: Dec. 13, 2004

(51) Int. Cl. A61K 8/00; A61K 8/02

(52) U.S. Cl. 424/401; 510/117

(57) ABSTRACT

A cleansing article is provided which includes an effervescent cleanser composition held within a rupturable packet and the packet is held within a porous receptacle. The effervescent cleanser composition is an intimate mixture of an acid material such as citric acid, an alkaline material such as sodium bicarbonate, and a surfactant.

### BACKGROUND OF THE INVENTION

1. Field of the Invention

The present invention relates to a personal skin care and cleansing implement and specifically one that contains a rupturable packet containing an effervescent cleansing composition.

2. The Related Art

Implements are frequently used to efficiently apply skin care and cleansing compositions and have included woven and nonwoven wipes, polymeric mesh bags and sponges to name a few widely used forms of implements.

An early example of cleansing pad technology is found in U.S. Pat. No. 1,808,834 (Busch Sr.). A fabric pouch is disclosed surrounding a cleansing composition mainly consisting of calcium and sodium carbonate.

U.S. Pat. NO. 4,234,442 (Cornelissens) describes a sachet which can consist of a water permeable material filled with an acidic and an alkaline constituent. Adipic, succinic and glutaric acids exemplify the acidic constituent. Sodium bicarbonate and carbonate represent the alkaline ingredient.

U.S. Pat. No. 4,272,393 (Gergely) describes a cleaning article formed of a porous flexible substrate, especially a cellulosic paper, impregnated with detergent and a gas-generating system. The latter is formed by separating an acidic component such as citric acid from a basic component such as sodium carbonate in two separate areas of the substrate.

U.S. Pat. No. 4,515,703 (Haq), U.S. Pat. No. 4,600,620 (Lloyd et al.) and U.S. Pat. No. 4,603,069 (Haq et al.) all describe wiping articles impregnated with surfactant. These do not contain any effervescent ingredients.

WO 97/43366 (Askew et al.) reports an effervescent system to improve dispensability of granular laundry detergent powders into wash water of automatic washing machines. Citric acid and bicarbonate combinations are employed to generate effervescence.

U.S. Pat. No. 6,063,390 (Farrell et al.); U.S. Pat. No. 6,217,854 (Farrell et al.); U.S. Pat. No. 6,610,311 (Charambura et al.); U.S. Pat. No. 6,610,312 (Farrell et al.); and U.S. Publication no. 2003/0003136 all describe effervescent cosmetic pillows. However the articles disclosed therein do not employ rupturable packetes to contain the effervescent cleansing compositions.

Surprisingly it has been found that a cleansing cloth containing an effervescent composition activated by contact with water can be provided where the effervescent composition is a stable flowable material contained in a unitary chamber and where the unitary chamber is ruptured and the powder contained therein can be activated with the addition of water.

### SUMMARY OF THE INVENTION

In one aspect of the invention is a cosmetic article for cleansing the skin and hair, the article including but not limited to:

a. a water permeable first substrate defining at least a portion of one wall of a receptacle;

b. a rupturable second substrate defining a unitary chamber positioned in the receptacle;

c. an anhydrous effervescent cleanser composition positioned within the unitary chamber, the composition including:

(i) from about 1 to about 80% of an alkaline material;

(ii) from about 0.5 to about 80% of an acid material;

(iii) from about 0.1 to about 30% of a surfactant; and wherein the alkaline and acid materials are blended together within the unitary chamber.

In another aspect of the invention is a cosmetic article for cleansing body surfaces, the article comprising a substantially sealed pillow plumped by effervescent generated carbon dioxide and exuding lather and an emollient through a water permeable first substrate wherein the article contains a second substrate defining a unitary chamber positioned in the pillow, the chamber containing an effervescent composition, and the second substrate having a rupturable portion.

In a further aspect of the invention is a method for cleansing skin including but not limited to the steps of:

a. wetting with water a cosmetic cleansing article,

b. generating foam from the article and

c. wiping skin surfaces with the wetted article, wherein the article includes:

1. a water permeable first substrate defining at least a portion of one wall of a receptacle;

2. a rupturable second substrate defining a unitary chamber positioned in the receptacle;

3. an anhydrous effervescent cleanser composition positioned within the unitary chamber, the composition including:

(i) from about 1 to about 80% of an alkaline material;

(ii) from about 0.5 to about 80% of an acid material;

(iii) from about 0.1 to about 30% of a surfactant; and wherein the alkaline and acid materials are blended together within the unitary chamber.

### BRIEF DESCRIPTION OF THE DRAWINGS

FIG. 1 is a perspective diagram of a preferred embodiment of the cleansing article of the invention, parts being broken away for clarity.

FIG. 1A is a perspective diagram of a second preferred embodiment of the cleansing article of the invention, parts being broken away for clarity.

FIG. 2 is a vertical cross-sectional view of FIG. 1 taken along line 2-2.

FIG. 3 is a detailed vertical cross-sectional view of FIG. 1 taken along line 3-3 showing a portion of the unitary chamber being received by a portion of the water permeable substrate.

### DETAILED DESCRIPTION OF THE INVENTION

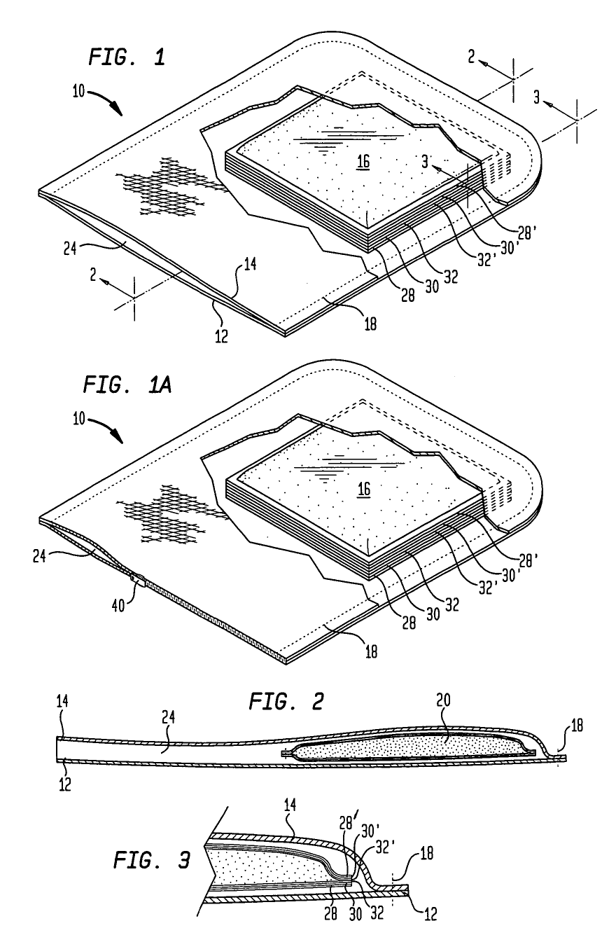

Now referring to the figures where like numbers represent like features, cosmetic article 10 is composed of first sheet 12 and second sheet 14, that are each individually water permeable first substrates attached via stitching 18 to form receptacle 24. Receptacle 24 is configured to receive packet 16. Packet 16 is composed of rupturable second substrates including a top three-layer rupturable laminate sheet composed of layers 28, 30, 32 and a bottom three-layer rupturable laminate sheet composed of layers 28′, 30′, 32′. Effervescent cleansing composition 20 is heat-sealed within the top laminate sheet and the bottom laminate sheet. In a further preferred embodiment illustrated in FIG. 1A, receptacle 24 employs sealing means such as zipper 40 that can be opened and closed in repeatable fashion by the user (i.e. reversible) to easily allow replacement of packet 16 with a fresh packet after packet 16 has been exhausted with use. In place of zipper 40, any suitable permanent or reversible closure method such as heat sealing, spot welds, needle punching, stitching, interlocking closure strips or points, Velcro® hooks and loops, buttons and the like may be used singly or in any combination. In another preferred embodiment, packet 16 may be attached to sheet 12 and/or second sheet 14 via any suitable attachment means to form a single-use disposable cosmetic article 10 where packet 16 is not readily replaceable after the cosmetic article has been used.

Laminate layers 32 and 32′ are each 0.2 mil (5 micron) thick polyethylene seal layers that have been heat sealed to each other along the periphery of packet 16.

Laminate layers 28 and 28′ are each 2.1 mil (53.3 micron) thick polyethylene. Laminate layers 32 and 32′ are thin enough that they can be readily breached by application of finger pressure on flowable cleansing composition 20 forcing the composition towards the heat seal between layers 32 and 32′ and rupturing layer 32, 32′ or both as sufficient pressure is supplied by the user. Laminate layers 30 and 30′ are each 0.2 mil (5 microns) thick polyethylene/butylene blend layers, i.e. a mixed resin formed from mixing a resin of the same kind as the inner surface of the packet (i.e. layers 32 and 32′) with a resin incompatible with the resin of the inner surface of the packet such as polybutylene. After layers 32, 32′ or both are breached by application of force by the user, layer 30, 30′ or both will delaminate from layers 32, 32′ or both as the case may be in the immediate vicinity of the breach, allowing the cleanser composition 20 to eventually exit packet 16 between the delamination of the layers

In one aspect of the invention is a cosmetic article for cleansing the skin and hair, the article including but not limited to:

a. a water permeable first substrate defining at least a portion of one wall of a receptacle (preferably where the receptacle is closable or sealable);

b. a rupturable second substrate defining a unitary chamber positioned in the receptacle;

c. an anhydrous effervescent cleanser composition positioned within the unitary chamber, the composition including:

(i) from about 1 to about 80% of an alkaline material;

(ii) from about 0.5 to about 80% of an acid material;

(iii) from about 0.1 to about 30% of a surfactant; and wherein the alkaline and acid materials are blended together within the unitary chamber. (preferably wherein the effervescent cleanser composition is sealed within the unitary chamber).

Preferably the inventive includes a cleanser composition that is sufficiently flowable under finger pressure applied by a user to rupture a portion of the second substrate. Advantageously the water permeable first substrate is selected from woven and nonwoven materials, and other natural or synthetic polymeric materials or blends thereof (such as apertured formed thermoplastic films, apertured plastic films, and hydroformed thermoplastic films; mesh composed of polymeric filaments (e.g. diamond mesh netting, etc.), natural sponge, porous foams; reticulated foams (e.g. polyurethane, regenerated cellulose, etc.); reticulated thermoplastic films; or thermoplastic scrims). Preferably the water permeable substrate is a fibrous substrate.

Advantageously the rupturable substrate has at least one feature selected from a weakened resin seal, a weakened membrane zone, a cutting or breaching member positioned to breach the rupturable substrate or any combination of the foregoing. Preferably the force necessary to release the contents of the unitary chamber is in the range of about 700 to 1200 gms/inch and more preferably about at least about 900 gms/inch.

In a preferred embodiment, the effervescent cleanser composition further comprises from about 0.01 to about 30% by weight of a skin benefit agent selected from the group consisting of emollients, anti-aging actives, antibacterials and fungicides, skin lighteners, sunscreens or mixtures thereof. Preferably the anti-aging actives are selected from the group consisting essentially of vitamins, retinoids or mixtures thereof. More preferably the cleanser composition further comprises about 0.1 to about 3% by weight of an emotive agent selected from the group consisting of fragrance, botanical extracts or mixtures thereof. Advantageously the emotive agent is incorporated into the effervescent cleanser composition by spray application onto one or more other components of the composition.

In a further preferred embodiment the water permeable first substrate is non-woven rayon and the surfactant in the cleanser composition includes sodium cocoyl isethionate, the acid includes citric acid and cleanser composition further comprises about 0.01 to about 1 % by weight of a skin deposition aid which is a cationic monomer or polymer.

In another aspect of the invention is a cosmetic article for cleansing body surfaces, the article comprising a substantially sealed pillow plumped by effervescent generated carbon dioxide and exuding lather and an emollient through a water permeable first substrate wherein the article contains a second substrate defining a unitary chamber positioned in the pillow, the chamber containing an effervescent composition, and the second substrate having a rupturable portion.

In a further aspect of the invention is a method for cleansing skin including but not limited to the steps of:

a. wetting with water a cosmetic cleansing article,

b. generating foam from the article and

c. wiping skin surfaces with the wetted article, wherein the article includes:

1. a water permeable first substrate defining at least a portion of one wall of a receptacle;

2. a rupturable second substrate defining a unitary chamber positioned in the receptacle;

3. an anhydrous effervescent cleanser composition positioned within the unitary chamber, the composition including:

(i) from about 1 to about 80% of an alkaline material;

(ii) from about 0.5 to about 80% of an acid material;

(iii) from about 0.1 to about 30% of a surfactant; and wherein the alkaline and acid materials are blended together within the unitary chamber.

Rupturable Packet:

The rupturable unitary chamber or packet may include a rupturable seal to allow a user to rupture the seal by squeezing or otherwise applying pressure to the contents resulting in the release of the contents from the packet. Such rupturable seals may include pealable seals, heat seals, pressure sensitive adhesive seals, mechanical interlocking seals, combinations of the foregoing and equivalents thereof. Alternatively, the rupturable packet may include weakened portions in the packet material itself such as scores, perforations, thin areas and the like. Also useful in the invention are external or internal cutting means such as pull tabs (optionally having sharp appendages such as metal shavings) or other items that may puncture the rupturable packet upon the application of pressure or tension, or may include any other means of rupturing a packet known in the art and equivalents thereof. Any combination of a rupturable seal, packet and/or cutting device may also be used. Preferably a rupturable seal or other system substantially impermeable to water vapor is used. Rupturable membranes are herein defined as to not include water soluble or dispersible membranes.

As discussed above, one useful rupturable seal embodiment takes advantage of the characteristic that the heat adhesive strength between a synthetic resin sheet and a sheet of a different type of resin is small. These seals are generally formed by taking a synthetic resin sheet that is of a type which is different from the innermost layer of the base material sheet and placing this different sheet between the base material sheets. This area is then heat sealed. Preferably this innermost layer which is adjacent to the effervescent composition is thin enough to rupture when moderate pressure is applied by a user of the article. When sufficient pressure is applied the flowable effervescent composition is propelled through the ruptured thin inner layer and then through the failed seal between the inner layer and the adjacent layer composed of a different type of resin. FIG. 3 illustrates an embodiment of this type of rupturable seal. Useful rupturable seals are described in e.g. U.S. Pat. No. 4,782,951 issued to Grasbach et al. on Nov. 8, 1988; U.S. Pat. No. RE 37,171 issued to Busche et al. on May 8, 2001; EP Patent No. 785066 B1 issued to Hergert et al. on Apr. 28, 2004; and EP patent no. 239319 B1 issued to Douglas et al. on Sep. 5, 1990 which are all incorporated by reference in their entirety.

The packet preferably has a length from about 4 cm to about 7 cm and a widith of about 3 cm to about 6 cm.

The packet of the present invention can be formed from a number of materials to make a single-layer or multi-layer film or sheet, preferably having flexibility. Examples of preferred materials to make the packet include the following: low-density polyethylene, middle-density polyethylene, high-density polyethylene, linear low-density polyethylene, polypropylene, polybutene-based resin, polymethylpentene-based resin, ionomer resin, ethylene-propylene copolymer, ethylene-vinyl acetate copolymer, ethylene- vinyl acetate copolymer saponified product, ethylene- acrylic acid copolymer, ethylene-ethyl acrylate copolymer, polyacrylic resin, polyacrylonitrile-based resin, polyester-based resin (e.g., polyethylene terephthalate, polyethylene naphthalate), polyamide-based resin, polystyrene-based resin, polyvinyl chloride-based resin, polyvinylidene chloride-based resin, polycarbonate-based resin, fluorine- based resin, phenolic resin, blends thereof and the like.

The Effervescent Cleansing System

Cosmetic wiping articles of the present invention when contacted with water advantageously billow to many times (preferably more than 10 but more preferably more than 40 times) their dry size when activated by water. The effervescent cleansing system exudes copious amounts of lather. A plumped “pillow” arises from the effervescent action. By careful control of the acidic and alkaline components, a squeaky clean rinsed feeling is felt on a user's skin.

A first essential component of compositions within the packet is that of an acidic material. Suitable for this purpose are any acids present in dry solid form. Especially appropriate are C₂-C₂₀ organic mono- and poly- carboxylic acids and especially alpha- and beta- hydroxycarboxylic acids; C₂-C₂₀ organophosphorus acids such as phytic acid; C₂-C₂₀ organosulfur acids such as toluene sulfonic acid; and peroxides such as hydrogen peroxide. Typical hydroxycarboxylic acids include adipic, glutaric, succinic, tartaric, malic, maleic, lactic, salicylic and citric acids as well as acid forming lactones such as gluconolactone and glucarolactone. Most preferred is citric acid. Also suitable as acid material may be encapsulated acids. Typical encapsulating material may include water soluble synthetic or natural polymers such as polyacrylates (e.g. encapsulating polyacrylic acid), cellulosic gums, polyurethane and polyoxyalkylene polymers. By the term “acid” is meant any substance which when dissolved in deionized water at 1 % concentration will have a pH of less than 7, preferably less than 6.5, optimally less than 5. These acids preferably at 25° C. are in solid form, i.e. having melting points no less than 25° C. Concentrations of the acid should range from about 0.5 to about 80%, preferably from about 10 to about 65%, optimally from about 20 to about 45% by weight of the total composition.

A second essential component of compositions within the packet is that of an alkaline material. The alkaline material is a substance which can generate a gas such as carbon dioxide, nitrogen or oxygen, i.e. effervesce, when contacted with water and the acidic material. Suitable alkaline materials are anhydrous salts of carbonates and bicarbonates, alkaline peroxides (e.g. sodium perborate and sodium percarbonate) and azides (e.g. sodium azide). Preferably the alkaline material is sodium or potassium bicarbonate. Amounts of the alkaline material may range from about 1 to about 80%, preferably from about 5 to about 49%, more preferably from about 15 to about 40%, optimally from about 25 to about 35% by weight of the total composition.

The effervescent cleansing composition contained in the packet is anhydrous so as to prevent or minimize any gas generation prior to use. By the term “anhydrous” is meant the presence of no more than about 10,8,7,5, 3.5 and optimally no more than about 1% water by weight of the total composition. Water of hydration is not considered to be water for purposes of the anhydrous definition. However, it is preferred to minimize, preferably to eliminate any water of hydration.

Advantageously the combined amount of acidic and alkaline materials will be at least about 1.5%, preferably from about 40 to about 95%, optimally from about 60 to about 80% by weight of the total composition.

A third necessary component of compositions according to the present invention is that of a surfactant, preferably a dry surfactant. Dry is as defined above. A suitable surfactant for the present invention is sodium cocoyl isethionate. Other useful surfactants include sodium methyl cocoyl taurate and sodium lauryl sulfate. Surfactants may be of the anionic, cationic, nonionic, amphoteric, zwitterionic varieties and combinations thereof. Amounts of the dry surfactant may range from about 0.1 to about 30%, preferably from about 1 to about 30%, optimally from about 8 to about 15% by weight of the total composition.

Other Components:

A variety of skin benefit agents may be included to improve afterfeel properties. Advantageously these substances will be available as anhydrous dry powders. Alternatively these substances may be flowable materials or liquids deposited upon or into a powdered substrate (e.g. sodium bicarbonate or zeolite) to achieve a resultant dry flowing liquid, semisolid or powder. Within the skin benefit agent scope are several categories of materials. These include emollients, antiaging actives, antibacterials and fungicides, skin lighteners, sunscreens and combinations thereof. Amounts of the skin benefit agents may range from about 0.001 to about 30%, preferably from about 0.1 to about 20%, more preferably from about 0.5 to about 10%, optimally between about 1 and about 5% by weight of the total composition.

Emollients may be in the form of natural or synthetic esters, silicone oils, hydrocarbons, starches, fatty acids and mixtures thereof. Typically the emollient may range in concentration from about 0.1 to about 35% by weight of the total composition.

Silicone oils may be divided into the volatile and nonvolatile variety. The term “volatile” as used herein refers to those materials that have a measurable vapor pressure at ambient temperature. Volatile silicone oils are preferably chosen from cyclic or linear polydimethylsiloxanes containing from 3 to 9, preferably from 4 to 5, silicon atoms.

Linear volatile silicone materials generally have viscosities less than about 5 centistokes at 25° C. while cyclic materials typically have viscosities of less than about 10 centistokes.

Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to about 100,000 centistokes at 25° C. Among the preferred non-volatile emollients useful in the present compositions are the polydimethyl siloxanes having viscosities from about 10 to about 400 centistokes at 25° C.

Among the ester emollients are:

(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.

(2) Ether-esters such as fatty acid esters of ethoxylated fatty alcohols.

(3) Polyhydric alcohol esters. Ethylene glycol mono and di-fatty acid ester, diethylene glycol mono- and di-fatty acid esters, polyethylene glycol (200-6000) mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.

(4) Wax esters such as beeswax, spermaceti, myristyl myristate, stearyl stearate and arachidyl behenate.

(5) Sterols esters, of which cholesterol fatty acid esters are examples thereof.

(6) Triglycerides such as sunflower seed oil, maleated sunflower seed oil, borage seed oil and safflower oil.

Hydrocarbons suitable as emollients include petrolatum, mineral oil, isoparaffins and hydrocarbon waxes such as polyethylene.

Starches are also suitable emollients. Typical of this class is tapioca and arabinogalactan.

Fatty acids may also be suitable as emollients. The fatty acids normally have from 10 to 30 carbon atoms. Illustrative of this category are pelargonic, lauric, myristic, palmitic, stearic, isostearic, hydroxystearic, oleic, linoleic, riconleic, arachidic, behenic and erucic acids.

Antiaging actives are also useful as skin benefit agents. Included within this category are vitamins, retinoids and combinations thereof. Amounts of these materials may range from about 0.001 to about 20% by weight of the total composition. Suitable vitamins include ascorbic acid, Vitamin B₆, Vitamin B₁₂, tocopherol as well as salts and C₁-C₂₀ esters thereof. Suitable retinoids include retinoic acid as well as its C₁-C₂₂ esters and salts, retinol and C₁-C₂₂ fatty esters of retinol including retinyl linoleate.

Another class of antiaging actives are the alpha- and beta- hydroxycarboxylic acids and salts thereof. Representative of this group are glycolic acid, lactic acid, malic acid, hydroxyoctanoic acid and mixtures of these as well as their salts. Suitable salts are the alkalimetal, ammonium and C₁-C₁₀ alkanol ammonium salts.

Antibacterials and fungicidals may also be included as skin benefit agents. Representative of these categories are triclosan, tricloban, hexetidene, chlorhexadene, gluconates, zinc salts (e.g. zinc citrate and zinc phenolsulfonate) and combinations thereof.

Skin lighteners may also be included under the skin benefit agents. Typical of this category are niacinamide, kojic acid, arbutin, vanillin, ferulic acid and esters thereof, resorcinol, hydroquinone, placental extract and combinations thereof.

Sunscreens may also be included as skin benefit agents. Particularly preferred are such materials as ethylhexyl p-methoxycinnamate, available as Parsol® MCX, and benzophenone-3, also known as Oxybenzone. Inorganic sunscreen actives may be employed such as microfine titanium dioxide, polyethylene and various other polymers. Amounts of the sunscreen agents will generally range from 0.1 to 30%, preferably from 2 to 20%, optimally from 4 to 10% by weight.

Adjunct functional agents may also be incorporated into compositions of the present invention. These include electrolytes, thickeners and mixtures thereof. Amounts of these substances may range from about 0.1 to about 20%, preferably from about 0.3 to about 10%, optimally between about 0.5 and about 5% by weight of the total composition.

Electrolytes may be selected from alkali, alkaline earth or ammonium salts of phosphates, silicates, halides, sulphates and mixtures thereof. Typical phosphates are potassium polymetaphosphate, sodium tripolyphosphate, sodium tetrapyrophosphate, sodium or potassium pyrophosphate and sodium hexametaphosphate. Most preferred is potassium polymetaphosphate available as Lipothix 100B® which is a 70:30 mixture of potassium polymetaphosphate and sodium bicarbonate, available from Lipo Chemicals, Inc., Paterson, N.J. Preferred sulphates are the magnesium sulphates.

Thickeners which may improve afterfeel properties on skin include inorganic or organic substances. A particularly preferred inorganic thickener is sodium magnesium silicate commercially available as Optigel SH®. Organic thickeners include alginic acid as well as sodium and calcium alginates, sodium carboxymethyl cellulose, hydroxypropyl methylcellulose, hydroxypropyl cellulose, hydroxyethyl cellulose and combinations thereof. Most preferred is alginic acid commercially available as Kelacid® from Sud-Chemie Rheologicals, Louisville, Ky. Alginic acid is highly effective at removing the slimy feel associated with deposits of alkaline material which are not fully rinsed away from the skin. Amounts of the thickener may range from about 0.1 to about 20%.

Polysaccharides useful in this invention are dry solid anhydrous substances such as sorbitol, sugars, (such as trehalose) starches, modified starches (e.g. aluminum octenyl succinate) and mixtures thereof. Most preferred is sorbitol.

Deposition aids may also be incorporated in compositions of the present invention. These assist in depositing skin benefit agents onto the skin surface. Particularly effective are cationic monomers and polymers for this purpose. Illustrative are the following: Lauryltrimethylammonium chloride (Laurtrimonium chloride); Stearyltri(2-hydroxyethyl)ammonium chloride (Quaternium-16); Lauryldimethylbenzylammonium chloride (Lauralkonium chloride); Oleyldimethylbenzylammonium chloride (Olealkonium chloride); Dilauryldimethylammonium chloride (Dilauryldimonium chloride); Cetyldimethylbenzylammonium chloride (Cetalkonium chloride); Dicetyldimethylammonium chloride (Dicetyldimonium chloride); Laurylpyridinium chloride (Laurylpyridinium chloride); Cetylpyridinium chloride (Cetylpyridinium chloride); N-(soya alkyl)-N,N,N-trimethylammonium chloride (Soyatrimonium chloride); Polydiallyidimethylammonium chloride (Polyquaternium-6); Diallydimethylammonium salt copolymerized with acrylamide (Polyquaternium-7); Guar hydroxypropyltrimonium chloride (Guar hydroxypropyltrimonium chloride); Copolymer of N-vinyl-pyrrolidone and N,N-dimethylaminoethylmethacrylate quaternized with dimethylsulfate (Polyquaternium-11); Copolymer of acrylamide and N,N-dimethylaminoethyl methacrylate,quaternized with dimethyl sulfate (Polyquaternium-5); Cationic hydroxyethylcellulosics (Polyquaternium-10); Cationic hydroxyethylcellulosics (Polyquaternium-24); Cetyltrimethylammonium chloride (Cetrimonium chloride); Decyldimethyloctylammonium chloride (Quaternium-24); Myristyltrimethylammonium chloride (Mytrimonium chloride); Polyoxyethylene (2)-cocomonium chloride (PEG-2 Cocomonium chloride); Methylbis(2-hydroxyethyl)cocoammonium chloride (PEG-2 Cocoyl Quaternium-4); Methylpolyoxyethylene-(15) cocoammonium chloride (PEG-15 Cocoyl Quaternium-4); Methylbis(2-hydroxyethyl) octadecyl ammonium chloride (PEG-2 Stearyl Quaternium-4); Methylpolyoxyethylene-(15) octadecylammonium chloride(PEG-15 Stearyl Quaternium-4); Methylbis(2-hydroxyethyl)-oleylammonium chloride (PEG-2 Oleyl Quaternium-4); Methylpolyoxyethylene-(15)oleylammonium chloride (PEG-15 Oleyl Quaternium-4), The names in parenthesis are given by the Cosmetic, Toiletry and Fragrance Association, Inc. in the CTFA Cosmetic Ingredient Dictionary. Most preferred for purposes of this invention are cationic guar gums such as Jaguar C13S® which is guar hydroxypropyltrimonium chloride. Amounts of the deposition aid may range from about 0.01 to about 1%, preferably from about 0.05 to about 0.5%, optimally from about 0.1 to about 0.3% by weight.

Advantageously an emotive agent such as a fragrance and/or botanical extract are included with the effervescent cleansing composition. Amounts of the fragrance and/or botanicals present in the effervescent cleansing composition may be at levels from abut 0.1 to about 3%, preferably from 0.5 to 2%, optimally from 0.8 to 1.5% by weight of the total composition.

The term “fragrance” is defined as a mixture of odoriferous components, optionally mixed with a suitable solvent diluent or carrier, which is employed to impart a desired odor. Particular preferred odoriferous components are cyclic and acyclic terpenes and terpenoids. These materials are based upon isoprene repeating units. Examples include alpha and beta pinene, myrcene, geranyl alcohol and acetate, camphene, dl-limonene, alpha and beta phellandrene, tricyclene, terpinolene, allocimmane, geraniol, nerol, linanool, dihydrolinanool, citral, ionone, methyl ionone, citronellol, citronellal, alpha terpineol, beta terpineol, alpha fenchol, borneol, isoborneol, camphor, terpinen-1-ol, terpin-4-ol, dihydroterpineol, methyl chavicol, anethole, 1,4 and 1,8 cineole, geranyl nitrile, isobornyl acetate, linalyl acetate, caryophyllene, alpha cedrene, guaiol, patchouli alcohol, alpha and beta santalol and mixtures thereof. Botanicals of particular use in the present invention include yarrow, chamomile, jasmine, lavender, horse chestnut, sage, thyme, yucca, coltsfoot and mixtures thereof.

Preservatives can desirably be incorporated into the cosmetic compositions of this invention to protect against the growth of potentially harmful microorganisms. Suitable traditional preservatives for compositions of this invention are alkyl esters of para-hydroxybenzoic acids. Other preservatives which have more recently come into use include hydantoin derivatives, propionate salts, and a variety of quaternary ammonium compounds. Cosmetic chemists are familiar with appropriate preservatives and routinely choose them to satisfy the preservative challenge test and to provide product stability. Particularly preferred preservatives are phenoxyethanol, methyl paraben, propyl paraben, imidazolidinyl urea, sodium dehydroacetate and benzyl alcohol. The preservatives should be selected having regard for the sue of the composition and possible incompatibilities between the preservatives and other ingredients in the emulsion. Preservatives are preferably employed in amounts ranging from 0.01 % to 2% by weight of the composition.

Natural vegetable materials from renewable resources are often desirable in cosmetic compositions. For instance, cosmetic compositions of the present invention may include beta-glucan derived from oats, commercially available under the trademark Microat SF from Nurture Inc., Missoula, Mont.

Colorants may also be included in compositions of the present invention. These substances may range from about 0.05 to about 5%, preferably between 0.1 and 3% by weight.

As discussed above, effervescent cleansing compositions of this invention will be placed within a packet formed of a rupturable second substrate wherein the packet will be disposed inside a receptacle formed advantageously between a first and second sheet comprised of the first substrate, preferably at least one of these being a flexible sheet. At least one of the sheets must have a portion that is water permeable, most preferably both sheets should have portions that have water permeability. For definitional purposes, first and second sheets can be folded-over panels of a single unitary sheet. Suitable materials for forming sheets may be rayon, polyester, polyethylene, polypropylene, cotton or any combination thereof. These sheets may be woven or non-woven. Most preferred is a non-woven rayon. Cellulosic paper fiber substrates are best not employed because of their insufficient wet-strength although they may be blended with other fibers referenced above; it is important that the first substrate sheets are not readily torn open through consumer rubbing of the article. Unlike laundry sachet articles, the receptacles composed of the first substrate of the present invention should not rupture to allow dispersion of the granular contents contained in the packet to spill undissolved into the wash water. Rather it is intended for all cleanser composition components to exit by dissolution through the permeable walls of the receptacle after being released from the packet.

Skin surfaces against which articles of the present invention are useful include face, body, scalp, axilla and even legs/feet. When the article is a foot cleanser, it would be advantageous for the pouch on one of its sides to be coarse while the second of the sheets may be soft and gentle. An abrasive non-woven flexible sheet in a foot cleanser product is useful for rubbing against calluses while the second sheet of the pouch remains smooth.

Articles according to the present invention may be formed in the following manner. In one aspect, constituents of the effervescent cleansing composition are placed into a dry mill or similar apparatus and blended until a uniformly distributed powder results. Thereafter, fragrance/botanical component is optionally sprayed into the dry mill with concurrent agitation of the powdered composition. The composition is then dispensed into a rupturable packet and the packet is sealed using art recognized and other equivalent techniques such as thermal sealing, adhesive sealing, ultrasonic sealing and the like. If the powdered composition is not flowable, then physical means such as a cutter or pull tab and the like is required to rupture the packet. Alternatively, in another embodiment of the invention, an anhydrous liquid or semisolid medium that is flowable under finger pressure supplied by a user is blended with the powder effervescent composition to form a homogenous blend and then the resulting blend is sealed within a rupturable packet.

In one preferred embodiment, a continuous roll of first sheet of the first substrate is unwound from a source roll over a moving conveyer belt. The effervescent cleansing composition rupturable packets are placed onto the sheet in a predetermined sequence. A second sheet of the first substrate is then in register placed over that of the packet loaded first substrate sheet. At this point the periphery surrounding the packet is tacked or sealed in register trapping the effervescent cleansing composition packet within. Cutters then separate one tacked or sealed section from another thereby forming the cosmetic article. One or more of the cosmetic articles are then packaged within a moisture impermeable outer package such as a laminated foil bag to prevent activation of the effervescent system during storage.

Ultrasonic welding may be employed as an alternative to heat-sealing of the first and second sheets of the first substrate together. Thread stitching, glue application or other closure mechanisms may also be utilized, especially for cleansing articles that will only be used once.

In a second preferred embodiment, the process above is carried out except that an opening is left so that fresh packets can be added to the receptacle to replace an expended packet. In a further preferred embodiment, a closure means is attached to the opening to allow the user to open and close the closure means at will to facilitate replacing expended packets with an unused packet.

The invention will now be described in greater detail by way of the following non-limiting examples. The examples are for illustrative purposes only and not intended to limit the invention in any way.

Except in the examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

All percentages in the specification and examples are intended to be by weight unless stated otherwise.

### EXAMPLE 1

An effervescent cleansing composition may be prepared according to the formulation reported in Table I. Phase A may be dry blended in a high speed shearing 5 mixer. Fragrance may then be sprayed onto the resultant powder as a Phase B. Three grams of the resultant powder are then placed into a 1.5 inch by 2.5 inch rupturable packet which is then heat sealed. The rupturable packet may have a pull tab adhered to a weakened area of the packet which when pulled causes the contents of the packet to become exposed. The sealed packet is then placed inside a 2 inch by 3 inch receptacle formed of non-woven rayon. All sides of the receptacle may be closed by double stitching with thread while allowing a length of pull tab to extend outside the receptacle.

TABLE I

| INGREDIENT | WEIGHT % |
|---|---|
| PHASE A | |
| Sodium Bicarbonate | 34.5 |
| Citric Acid (Anhydrous) | 40.4 |
| Sodium Cocoyl Isethionate (Powder) | 11.6 |
| Sodium Sesquicarbonate | 5.0 |
| Lipothix 100B ® (Potassium | 0.5 |
| Polymetaphosphate/Bicarbonate 70:30) | |
| Optigel SH ® (Sodium Magnesium Silicate) | 1.0 |
| Kelacid ® (Alginic Acid) | 1.0 |
| Sorbitol | 5.0 |
| PHASE B | |
| Fragrance | 1.0 |

### EXAMPLE 2

Another effervescent cleansing composition may be prepared according to the formulation reported in Table II using the procedure provided in example 1.

TABLE II

| INGREDIENT | WEIGHT % |
|---|---|
| PHASE A | |
| Sodium Bicarbonate | 32.3 |
| Citric Acid (Anhydrous) | 41.1 |
| Sodium Cocyl Isethionate (Powder) | 11.6 |
| Sodium Sesquicarbonate | 5.0 |
| Lipothix 100B ® (Potassium | 0.5 |
| Polymetaphosphate/Bicarbonate 70:30) | |
| Optigel SH ® (Sodium Magnesium Silicate) | 1.0 |
| Kelacid ® (Alginic Acid) | 1.0 |
| Sorbitol | 5.0 |
| Laracare A200 ® (Arabinogalactan) | 1.0 |
| Ascorbic Acid | 0.5 |
| PHASE B | |
| Fragrance | 1.0 |

### EXAMPLE 3

A face cleansing effervescent composition may be prepared according to the formulation reported in Table III using the procedure provided in example 1.

TABLE III

| INGREDIENT | WEIGHT % |
|---|---|
| PHASE A | |
| Sodium Bicarbonate | 33.6 |
| Citric Acid (Anhydrous) | 39.0 |
| Sodium Cocyl Isethionate (Powder) | 3.0 |
| Sodium Methyl Cocoyl Taurate | 6.0 |
| Sodium Lauryl Sulfate | 2.5 |
| Sodium Sesquicarbonate | 5.0 |
| Lipothix 100B ® (Potassium | 0.5 |
| Polymetaphosphate/Bicarbonate 70:30) | |
| Optigel SH ® (Sodium Magnesium Silicate) | 2.0 |
| Tapioca | 5.5 |
| Methyl Gluceth 20-Benzoate | 2.0 |
| Guar Hydroxypropyl Trimonium Chloride | 0.25 |
| PHASE B | |
| Fragrance | 0.65 |

### EXAMPLE 4

A still further effervescent cleansing composition according to the present invention may be prepared according to the formulation listed in Table IV. Phase A is prepared by dry mixing of the ingredients in a high speed shear mixer. Three grams of the resultant powder are suspended in a sufficient quantity of any suitable anhydrous liquid medium that does not react with the other ingredients of the cleansing composition such as mineral oil so as to disperse the powder without clumping. The resulting dispersion is then placed into a two inch by three inch rupturable packet formed of a laminate film composed of the following three layers arranged in order of outermost to innermost that is adjacent to the cleansing composition: 2.1 mil polyethylene: 0.2 mil polyethylene/butylene blend: 0.2 mil polyethylene and the packet is sealed. The packet is then placed within a slightly larger woven or non-woven receptacle spot welded on three sides through a zippered opening on a fourth side. The zipper is then closed. Application of finger pressure on the sealed packet by the user through the woven or non-woven layer will cause the composition to nearly simultaneously breach the 0.2 mil polyethylene layer and separate the weakened heat seal between that layer and the polyethylene/butylene blend layer, thus exposing the packet contents to water applied by the user. The porosity of the woven or non-woven receptacle containing the packet is sufficient to allow transfer of dissolved ingredients while small enough to allow the article to become plump due to the gas produced by the effervescence.

TABLE IV

| INGREDIENT | WEIGHT % |
|---|---|
| PHASE A | |
| Potassium Bicarbonate | 29.5 |
| Lactic Acid (Anhydrous) | 45.4 |
| Sodium Sulfosuccinate | 11.6 |
| Sodium Sesquicarbonate | 5.0 |
| Lipothix 100B ® (Potassium | 0.5 |
| Polymetaphosphate/Bicarbonate 70:30) | |
| Optigel SH ® (Sodium Magnesium Silicate) | 1.0 |
| Kelacid ° (Alginic Acid) | 1.0 |
| Sorbitol | 5.0 |
| PHASE B | |
| Fragrance | 1.0 |
| Licorice Extract | 0.1 |

### EXAMPLE 5

Still another effervescent cleansing composition may be prepared according to the formulation listed in Table V according to the method of Example 4. The ingredients are dry blended in a high speed shearing mixer. Fragrance and herbal extract are sprayed onto the powder and further blended to achieve homogeneity. Three grams of resultant powder dispersed in an anhydrous fluid are placed into a two inch by three inch packet formed of a laminate film composed of the following three layers: 2.1 mil polyethylene: 0.2 mil polyethylene/butylene blend: 0.2 mil polyethylene and the packet is sealed. The sealed packet is itself sealed in a non-woven polypropylene receptacle via convection heat sealing along the perimeter thereof resulting in a convenient, disposable article.

TABLE V

| INGREDIENT | WEIGHT % |
|---|---|
| PHASE A | |
| Sodium Bicarbonate | 29.5 |
| Citraconic Acid (Anhydrous) | 45.4 |
| Methyl Glucamide | 11.6 |
| Sodium Sesquicarbonate | 5.0 |
| Lipothix 100B ® (Potassium | 0.5 |
| Polymetaphosphate/Bicarbonate 70:30) | |
| Optigel SH ® (Sodium Magnesium Silicate) | 1.0 |
| Kelacid ® (Alginic Acid) | 1.0 |
| Sorbitol | 5.0 |
| PHASE B | |
| Fragrance | 0.9 |
| Yarrow | 0.1 |

The compositions that are rendered flowable by dispersion in a suitable anhydrous liquid as provided in table I-V may be packed in any suitable plastic resinous film to form a rupturable packet such as a 3-layer laminate which is formed from the following materials listed from the outside proceeding inwardly that is adjacent to the cleansing composition: first, 2.1 mil white polyethylene, then 0.2 mil EZ Peel® blend and lastly 0.2 mil sealable polyethylene (Curwood Grade 4476-1 Protective Packaging film available from Curwood, Inc., Oshkosh, Wis.). The rupturable seal can be formed at a pressure of 30 psi applied for 1 second at 60° C. This will give the rupturable seal a burst strength of about 900 grams/inch at room temperature.

While performance will vary with different flexible sheet materials chosen for the packet, if the laminate referred to above is used the following properties are expected to be observed. The packet will have a moisture vapor transmission rate of less than about 0.5 grams water/100 sq. inches/24 hr at 24 C). and 90% relative humidity. The oxygen permeability of the same compartment will be about 200 cc/100 sq. inches/24 hr at 23° C. and 0 % relative humidity.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

What is claimed is:

1. A cosmetic article for cleansing the skin and hair, the article comprising:
a. a water permeable first substrate defining at least a portion of one wall of a receptacle;
b. a rupturable second substrate defining a unitary chamber positioned in the receptacle;
c. an anhydrous effervescent cleanser composition positioned within the unitary chamber, the composition including:
(i) from about 1 to about 80% of an alkaline material;
(ii) from about 0.5 to about 80% of an acid material;
(iii) from about 0.1 to about 30% of a surfactant; and wherein the alkaline and acid materials are blended together within the unitary chamber.

2. The article of claim 1 wherein the cleanser composition is sufficiently flowable under finger pressure applied by a user to rupture a portion of the second substrate.

3. The article of claim 1 wherein the water permeable first substrate is selected from woven and nonwoven materials, and other natural or synthetic polymeric materials or any combination of the foregoing.

4. The article of claim 1 wherein the water permeable substrate is a fibrous substrate.

5. The article of claim 1 wherein the rupturable substrate has at least one feature selected from a weakened resin seal, a weakened membrane zone, a cutting or breaching member positioned to breach the rupturable substrate or any combination of the foregoing.

6. The article of claim 1 wherein the force necessary to release the contents of the unitary chamber is in the range of about 700 to 1200 gms/inch.

7. The article of claim 1 wherein the effervescent cleanser composition further comprises from about 0.01 to about 30% by weight of a skin benefit agent selected from the group consisting of emollients, anti-aging actives, antibacterials and fungicides, skin lighteners, sunscreens or mixtures thereof.

8. The article according to claim 7 wherein the anti-aging actives are selected from the group consisting essentially of vitamins, retinoids or mixtures thereof.

9. The article of claim 1 wherein the effervescent cleanser composition further comprises about 0.1 to about 3% by weight of an emotive agent selected from the group consisting of fragrance, botanical extracts or mixtures thereof.

10. The article of claim 9 wherein the emotive agent is incorporated into the effervescent cleanser composition by spray application onto one or more other components of the composition.

11. The article of claim 1 wherein the water permeable first substrate is non-woven rayon.

12. The article of claim 1 wherein the surfactant includes sodium cocoyl isethionate.

13. The article of claim 1 wherein the acid includes citric acid.

14. The article of claim 1 wherein the effervescent cleanser composition further comprises about 0.01 to about 1% by weight of a skin deposition aid which is a cationic monomer or polymer.

15. A cosmetic article for cleansing body surfaces, the article comprising a substantially sealed pillow plumped by effervescent generated carbon dioxide and exuding lather and an emollient through a water permeable first substrate wherein the article contains a second substrate defining a unitary chamber positioned in the pillow, the chamber containing an effervescent composition, and the second substrate having a rupturable portion.

16. A method for cleansing skin comprising the steps of:
a. wetting with water a cosmetic cleansing article,
b. generating foam from the article and
c. wiping skin surfaces with the wetted article, wherein the article includes:
a water permeable first substrate defining at least a portion of one wall of a receptacle;
a rupturable second substrate defining a unitary chamber positioned in the receptacle;
an anhydrous effervescent cleanser composition positioned within the unitary chamber, the composition including:
(i) from about 1 to about 80% of an alkaline material;
(ii) from about 0.5,to about 80% of an acid material;
(iii) from about 0.1 to about 30% of a surfactant; and wherein the alkaline and acid materials are blended together within the unitary chamber.
